(19) United States
(12) Patent Application Publication
(10) Pub. No.: US 2026/0204409 A1
(43) Pub. Date: Jul. 16, 2026

(54) TCR Repertoire-Based Machine Learning for Colorectal Cancer Diagnosis

(71) Applicant: City University of Hong Kong, Hong Kong (CN)

(72) Inventors: Guan YANG, Hong Kong (CN); Gaochen ZHU, Hong Kong (CN)

(21) Appl. No.: 19/021,481

(22) Filed: Jan. 15, 2025

(51) Int. Cl. G16H 50/20; G16B 30/00; G16B 40/20; G16H 10/60

(52) U.S. Cl. CPC G16H 50/20; G16B 30/00; G16B 40/20; G16H 10/60

(57) ABSTRACT

A machine learning-(ML-)based method is developed for diagnosing colorectal cancer (CRC) based on sequences obtained from T cell receptor (TCR) sequencing of a peripheral blood TCR repertoire of a person. TRBV and TRBJ gene usages in the sequences are determined and combined to yield a plurality of TRBV-TRBJ combinations, which is a plurality of V-J gene combination features representing pairing of V and J gene segments of TCR β-chain of the TCR repertoire. A fraction indicating a relative abundance of an individual TRBV-TRBJ combination in the sequences is determined so as to form a TRBV-TRBJ gene combination fraction table for the plurality of TRBV-TRBJ combinations via pairing each TRBV-TRBJ combination with the fraction associated therewith. A ML model, such as a random forest model, is used to classify the fraction table into positive and negative classes. The positive class indicates that the person is diagnosed with CRC.

### ABBREVIATIONS

AdaBoost adaptive boosting
AUC area under the ROC curve
CDR3 complementarity determining region 3
CRC colorectal cancer
DT decision tree
FPR false positive rate
HEC high-expansion clone
KNN K-nearest neighbors
LR logistic regression
ML machine learning
RF random forest
ROC receiver operating characteristic
SVM support vector machine
TPR true positive rate
TCR T cell receptor

### TECHNICAL FIELD

This application generally relates to a ML technique for diagnosing CRC. Particularly, this application relates to a ML-based diagnosis model that utilizes peripheral blood TCR repertoire profiling to diagnose CRC.

### BACKGROUND

CRC ranks among the top three most commonly diagnosed cancers globally and continues to be a significant contributor to cancer-related deaths [1]. Despite decades of cancer research, the fundamental mechanisms responsible for the initiation and progression of this ubiquitous disease have not been fully elucidated [2]. Early diagnosis of CRC is crucial for ensuring appropriate treatment, as the cancer can spread to other organs, commonly the liver and lungs, leading to secondary tumors and complications [3]. However, current diagnostic approaches such as colonoscopy, fecal occult blood test, and imaging technology have limitations including the risk of perforation, false-positive results, missed non-bleeding lesions, and radiation side effects [4]. Therefore, there is an urgent need to explore and develop innovative diagnostic approaches for CRC.

In recent years, there has been significant interest in the role of the immune system in cancer, particularly with the advancements in immunotherapies. There is a direct correlation between the occurrence and progression of CRC and an aberrant immunological microenvironment, making immunotherapy a promising approach [5]. T lymphocytes play a crucial role in the immune response against tumors by recognizing tumor-associated antigens through specific receptors. The antigen-recognition part of the T cell membrane is composed of TCRs, which derive their specificity and diversity mainly from the variable CDR3 and the random rearrangement and junction region mutation of Variable (V), Diversity (D), and Joining (J) regions. Importantly, numerous studies have highlighted the significance of TCR and CDR3 diversity in cancer diagnosis, therapy, and prognosis [6-8]. Thus, performing peripheral blood repertoire profiling can be a promising diagnosis tool for CRC, as it reflects the dynamic changes in TCR repertoire that could serve as a biomarker to monitor the immunomodulatory process.

ML techniques have been proven successful in diagnosing and predicting various diseases, including cardiovascular diseases, cancer, and other immune-related disorders by analyzing the huge amount of microbiota data [9, 10]. However, gut microbiota-based ML methods for cancer diagnosis have shown limitations, including limited biological evidence supporting microbiome-phenotype associations [11] and a high misdiagnosis rate when applied to new communities [12]. In contrast, TCR-based ML methods can identify and predict tumor-associated antigens with high specificity, offering promising prospects for cancer diagnosis. In addition, this approach leverages the natural surveillance capabilities of the immune system against malignancies [13]. Therefore, it is desirable to investigate the diagnostic value of TCR repertoire-based ML in CRC.

### SUMMARY

In the present disclosure, the diagnostic value of TCR repertoire-based ML in CRC is confirmed by developing and experimentally verifying a ML model for CRC diagnosis based on sequence data obtained from TCR sequencing of a peripheral blood TCR repertoire. Specifically, the present disclosure provides a computer-implemented method for diagnosing CRC for a person.

The method comprises: receiving one or more sequences obtained from TCR sequencing of a peripheral blood TCR repertoire of the person; determining TRBV and TRBJ gene usages in the one or more sequences; combining the TRBV and TRBJ gene usages to yield a plurality of TRBV-TRBJ combinations for the TCR repertoire, the plurality of TRBV-TRBJ combinations being a plurality of V-J gene combination features representing pairing of V and J gene segments of TCR β-chain of the TCR repertoire; determining a fraction associated with an individual TRBV-TRBJ combination, wherein the fraction indicates a relative abundance of the individual TRBV-TRBJ combination in the one or more sequences; forming a TRBV-TRBJ gene combination fraction table for the plurality of TRBV-TRBJ combinations via pairing the individual TRBV-TRBJ combination with the fraction associated therewith; and using a ML model to classify the TRBV-TRBJ gene combination fraction table into a positive class and a negative class after the ML model is trained, the positive class indicating that the person is diagnosed with CRC, the negative class indicating that the person is not diagnosed with CRC.

In certain embodiments, the ML model is configured with an attention mechanism to focus on a plurality of selected TRBV-TRBJ gene combinations in processing the TRBV-TRBJ gene combination fraction table, wherein the plurality of selected TRBV-TRBJ gene combinations consists of combinations of: TRBV19 and TRBJ1-5; TRBV10-3 and TRBJ 1-1; TRBV2 and TRBJ1-2; TRBV29-1 and TRBJ 2-5; TRBV29-1 and TRBJ2-7; TRBV27 and TRBJ2-7; TRBV12-4 and TRBJ1-2; TRBV4-1 and TRBJ1-2; TRBV27 and TRBJ2-2; TRBV27 and TRBJ1-1; TRBV6-5 and TRBJ2-2; TRBV13 and TRBJ2-2; TRBV3-1 and TRBJ2-2; TRBV7-9 and TRBJ1-4; TRBV19 and TRBJ1-4; TRBV27 and TRBJ1-4; TRBV6-1 and TRBJ2-3; TRBV7-2 and TRBJ1-4; TRBV7-9 and TRBJ1-6; and TRBV27 and TRBJ1-3.

In certain embodiments, each of the received one or more sequences is a CDR3 sequence.

In certain embodiments, the ML model is a RF model.

In certain embodiments, the ML model is selected from a LR model, a KNN model, a Naive Bayes model, a DT model, a gradient boosting model and an AdaBoost model.

In certain embodiments, the ML model is a transformer.

The method may further comprise training the ML model.

A computing system for diagnosing CRC for a person is realizable based on the disclosed method. In particular, the computing system comprises one or more computers configured to execute a process of diagnosing CRC for the person according to any of the embodiments of the disclosed method.

Other aspects of the present disclosure are disclosed as illustrated by the embodiments hereinafter.

### BRIEF DESCRIPTION OF THE DRAWINGS

FIG. 1 is a schematic illustration of a pipeline workflow of TCR repertoire analysis, where the workflow includes the steps of sample collection, establishment of a library, TCR repertoire construction, establishment of a CRC diagnostic model and biological interpretation.

FIG. 2, which includes subplots A-D, provides a comprehensive analysis of TCR repertoire diversity, clonality, and differential Vβ and Jβ gene usage in CRC patients and healthy individuals, where: subplot A illustrates TCR Repertoire Shannon Diversity, each data point representing an individual sample; subplot B illustrates TCR repertoire Clonality, each data point representing an individual sample; subplot C depicts a Jβ gene usage volcano plot; and subplot D depicts a Vβ gene usage volcano plot (Wilcoxon test, p value <0.05 considered significant).

FIG. 3, which includes subplots A and B, provides TCR repertoire analysis between CRC patients and healthy individuals, where: subplot A provides a comparison of HEC number distribution between healthy controls and CRC patients, *p<0.05, Mann-Whitney U test; and subplot B depicts a Vβ gene usage volcano plot.

FIG. 4, which includes subplots A-F, illustrates performance of the RF models, where: subplot A plots the accuracy and AUC across five folds of cross-validation; subplot B plots the ROC curve for the test set with an AUC of 0.9798, in which the ROC curve plots the TPR against the FPR, demonstrating the model's diagnostic ability to distinguish between CRC and healthy individuals; subplot C plots The ROC curve for the external validation set with an AUC of 0.9357; subplot D depicts a confusion matrix for the test set, detailing the number of correct predictions (diagonal entries) versus incorrect predictions (off-diagonal entries); subplot E depicts the confusion matrix for the external validation set; and subplot F provides a comparative assessment of accuracy and AUC across training, test and external datasets.

FIG. 5, which includes subplots A and B, illustrates identification of key features distinguishing CRC patients from healthy controls, where: subplot A lists the top 20 feature importances determined by permutation importance for the CRC diagnostic model, in which important scores quantify the impact of each feature on the model's predictive accuracy, with higher values indicating greater importance; and subplot B depicts a heatmap illustrating the expression levels of the top 20 features between CRC and healthy individuals.

FIG. 6, which includes subplots A and B, provides performances of RF models employing different numbers of features in the test cohort, where: subplot A plots ROC curves for the test set with different numbers of features; and subplot B provides a comparative analysis of test set accuracy and AUC across ML models trained with different numbers of features.

FIG. 7 provides a statistical analysis of the top 20 features, where the top 20 features were analyzed using the Wilcoxon test (*p<0.05, **p<0.01, *p<0.001).

FIG. 8, which includes subplots A-C, provides performances of seven ML methods in TCR repertoire-based disease classification, where: subplot A plots the mean ROC curves for the validation sets across the seven ML methods; subplot B plots the ROC curves for the test set across the seven ML methods; and subplot C provides a comparative analysis of test set accuracy and AUC across the seven ML methods.

FIG. 9 depicts a flowchart showing exemplary steps of a computer-implemented method as disclosed herein for diagnosing CRC for a person.

Skilled artisans will appreciate that elements in the figures are illustrated for simplicity and clarity and have not necessarily been depicted to scale.

### DETAILED DESCRIPTION

As mentioned above, there exists a need to improve CRC diagnosis. Systemic characterization of the human TCR repertoire provides an opportunity to develop a better diagnosis of this major human disease. A major goal of the present disclosure is to diagnose CRC using ML-based algorithms and to identify the most effective one in terms of accuracy and precision.

In the present disclosure, specifically, a ML-based diagnosis model that utilizes peripheral blood TCR repertoire profiling to diagnose CRC is developed and tested. The developed model is validated by using publicly available TCR repertoire datasets, further strengthening the potential of the ML-based approach in cancer diagnostics. After the diagnostic model is detailed, embodiments of the present disclosure will be elaborated based on the disclosed details, examples, applications, etc. of the diagnostic model.

### 1. Diagnostic Model Development: Materials and Methods

In U.S. Provisional Patent Application No. 63/625,326 filed on Jan. 26, 2024, the disclosure of which is incorporated herein by reference in its entirety, the Inventors of the present disclosure disclose research results on CRC diagnosis using ML-based algorithms. Particularly, a k-mer frequency encoding of raw sequence data of a TCR repertoire was used. In an analysis of CDR3 sequences containing 39 types of 3-mers, the Inventors observed that the most abundant CDR3 sequences in almost all CRC samples (viz., samples of CRC patients) could be mapped to the TRBV7-9, TRBV28, TRBJ2-5, and TRBJ2-7 genes, all of which were significantly higher in the CRC group compared to the healthy control group. Furthermore, the Inventors observed substantial differences in the V-J combinations formed by these genes demonstrated between the two groups. For instance, a representative CRC sample and a healthy donor sample showed that the combinations of TRBV7-9 and TRBJ1-2, TRBV28 and TRBJ2-7, and TRBJ2-5 and TRBV11-2 were notably more abundant in the representative CRC sample than in the healthy control sample. This discrepancy supports potential clinical relevance of these gene combinations in CRC pathology. Based on the foregoing observations, in the present disclosure, the Inventors utilize V-J combinations in diagnosing CRC

### 1.1. Data Collection

The TCR repertoire data were retrieved from the Sequence Read Archive database available on the National Center for Biotechnology Information platform and the Genome Sequence Archive in the National Genomics Data Center. A total of 220 CRC cases were cataloged under the BioProject (PRJNA754274 [14], 83 samples; PRJCA009632 [15], 107 samples; PRJNA1049886, 30 samples). The 278 non-CRC cases were from BioProject (PRJNA754274 [14], 20 samples; PRJNA930724 [16], 22 samples; PRJEB40492, 46 samples; PRJEB50045 [17], 183 samples, and PRJNA821039 [18], 7 samples). Apart from the four samples within BioProject PRJNA821039 that are GATA2-deficient individuals, all remaining non-CRC samples are healthy controls. All CRC patients enrolled in this study were recruited from China. The control group consisted of individuals from China and several European countries, including the United Kingdom, Germany, and Switzerland.

### 1.2. Bioinformatics and Statistical Analyses for the TCR Repertoire

The raw sequence data underwent quality filtering using Trimmomatic (V.39) to eliminate adaptor sequences and low-quality reads (quality score <30). MiXCR (v.4.5.0) was employed to align the sequencing data to the reference sequences for the V, D, J, and Constant (C) gene segments of the TCR [19]. Following alignment, the data were assembled to determine the specific gene region sequences on the TCRβ, particularly the CDR3. After the initial processing with MiXCR, VDJtools (v.1.2.1) was harnessed for further analysis and manipulation of the resultant data including clonal numbers/types, repertoire richness/diversity, unique CDR3 species and frequency. The analysis of differences among the data groups was performed with the Wilcoxon test. A p value <0.05 was considered significant. The statistical analyses were conducted with R (v.4.3.1) and GraphPad Prism software (v.9.0.0).

### 1.3. Supervised Machine Learning

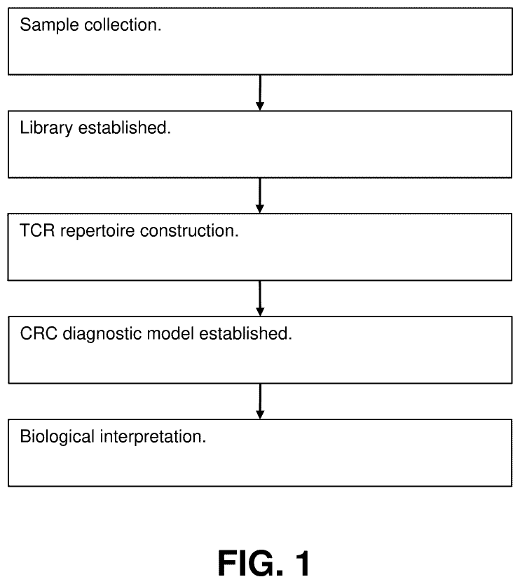

The data processing and analysis workflow is presented in FIG. 1. In the classification of CRC versus non-CRC, seven different supervised ML algorithms, which are RF, LR, KNN, DT, Naive Bayes, Gradient Boosting and AdaBoost, were trained with the features of TCR repertoire. CRC samples are labeled as positive control and non-CRC samples are labeled as negative control.

Among these algorithms, RF is a powerful ensemble learning method employed for classification tasks [24]. It builds multiple decision trees during training, with each tree constructed on a data sample extracted from a training set. The output of RF is the class selected from most trees for classification tasks. LR is a statistical method used for binary classification, modeling the relationship between dependent and independent variables to predict the probability of occurrence within a binary outcome [20]. KNN is a non-parametric method used for classification and regression, identifying the majority class of k-nearest neighbors to the query point in the feature space [21]. DT is a powerful and widely used classification model in data mining and ML, which efficiently partition data by recursively testing numeric features against threshold values [25]. Naive Bayes classification is a probabilistic model based on Bayes' theorem, assuming independence among predictors, which simplifies the computation of conditional probabilities [26]. Gradient Boosting is a ML technique that constructs an additive model in a forward stage-wise fashion. It iteratively trains a series of weak learners, typically decision trees, where each subsequent model focuses on fitting the residual errors of the preceding model [27]. AdaBoost is an iterative algorithm that combines multiple weak classifiers into a strong classifier. By adjusting the weights of training samples in each training, focusing on samples misclassified in the previous round, thereby improving the classification performance of the model [28].

A comprehensive review by Zhang et al. on comparing these algorithms across various biological classification tasks demonstrated that RF consistently outperforms other methods, particularly in handling complex biological data with high dimensionality and potential noise [29]. The review highlights RF's advantages in terms of robustness against overfitting, ability to handle missing values, and capacity to provide feature importance rankings, making it especially suitable for biological classification problems.

We analyzed the TRBV and TRBJ usages from each sample and combined them to create unique V-J gene combination features, each denoted as a TRBV-TRBJ combination which represents the pairing of V and J gene segments of the TCR β-chain. The fraction associated with each combination was used as a quantitative feature, representing the relative abundance of each V-J combination in the sample. We compiled all unique V-J combinations across all samples to form a comprehensive feature set. Each sample was transformed into a feature vector where each element corresponds to the fraction of a specific V-J combination. Missing values (i.e. combinations not present in a sample) were set to zero. The final input was a feature matrix X of dimension n×m, where n is the number of samples, and m is the number of unique V-J combinations identified across all samples. The label y was assigned as “CRC” for colorectal cancer samples and “HC” for healthy controls.

The categorical labels were encoded into numerical values using scikit-learn's LabelEncoder, transforming “CRC” and “HC” into binary labels (1 and 0, respectively). The dataset was then split into training and testing sets using a stratified 70/30 split to maintain the proportion of classes in both sets which is essential to ensure balanced representation of both classes in the training and testing datasets.

We employed stratified k-fold cross-validation with five folds (n_splits=5), ensuring that each fold had a similar class distribution. For each algorithm, hyperparameter tuning was performed to optimize model performance. We defined parameter grids and utilized scikit-learn's GridSearchCV for exhaustive search over specified parameter values. The models and their associated hyperparameters are as follows:
RF: Number of estimators (n_estimators), maximum depth (max_depth), maximum features (max_features), and class weights (class_weight).
LR: Regularization strength (C), penalty (penalty), and solver (solver).
KNN: Number of neighbors (n_neighbors) and weighting function (weights).
Naive Bayes: Since the Gaussian Naive Bayes classifier does not have hyperparameters that require tuning, it was used with default settings.
DT: Maximum depth (max_depth), minimum samples required to split (min_samples_split), and class weights (class_weight).
Gradient Boosting: Number of estimators (n_estimators), learning rate (learning_rate), and maximum depth (max_depth).
AdaBoost: Number of estimators (n_estimators) and learning rate (learning_rate).

More detailed model parameters are listed in Table 1. Each model was trained using the training folds, and performance was validated on the validation fold within the cross-validation framework. The hyperparameters yielding the highest mean AUC score during cross-validation were selected for each model.

TABLE 1
Parameters used in constructing different ML models.

| | | | | | Naive | Gradient | |
|---|---|---|---|---|---|---|---|
| | RF | LR | KNN | DT | Bayes | Boosting | AdaBoost |
| n_ | 100, 200, | / | / | / | / | 100, 200 | 50, 100, |
| estimators | 300 | | | | | | 200 |
| max_ | None, 10, | / | / | None, 5, 10 | / | 3, 5, 10 | / |
| depth | 20, 30 | | | | | | |
| max_ | ‘auto’, | / | / | / | / | / | / |
| features | ‘sqrt’, ‘log2’ | | | | | | |
| class_ | ‘None’, | / | / | ‘None’, | / | / | / |
| weight | ‘balanced’ | | | ‘balanced’ | | | |
| C | / | 0.01, 0.1, | / | / | / | / | / |
| | | 1, 10 | | | | | |
| penalty | / | ‘12’ | / | / | / | / | / |
| solver | / | ‘lbfgs’ | / | / | / | / | / |
| n_ | / | / | 3, 5, 7 | / | / | / | / |
| neighbors | | | | | | | |
| weights | / | / | ‘uniform’, | / | / | / | / |
| | | | ‘distance’ | | | | |
| min_ | / | / | / | 2, 5, 10 | / | / | / |
| samples_ | | | | | | | |
| split | | | | | | | |
| learning | / | / | / | / | / | 0.01, 0.1, 1 | 0.01, 0.1, 1 |
| rate | | | | | | | |

After hyperparameter tuning and model training, we evaluated the models on both the validation data during cross-validation and the independent test set. We computed the mean and standard deviation of accuracy and AUC across the five folds to assess the stability and generalization capability of each model. The best-performing model from the cross-validation (based on AUC) was evaluated on the held-out test set to assess its predictive performance on unseen data. For each model, we generated the ROC curves by plotting the true positive rate (sensitivity) against the false positive rate (1-specificity) at various threshold settings. The mean ROC curve for each model was calculated by interpolating the ROC curves from the cross-validation folds.

The analysis was conducted using Python (v.3.6.13) with the following libraries: pandas (v.1.1.5), NumPy (v.1.19.2), scikit-learn (v.0.24.2), Matplotlib (v.3.3.4).

### 1.4. Feature Importance Measures

To assess the impact of each feature on the classifier's predictive performance, we employed the permutation importance method within a cross-validation framework. Permutation importance is a model-agnostic technique that measures the decrease in model performance when the values of a single feature are randomly shuffled, thereby breaking the association between the feature and the target variable. This approach allows for an unbiased estimation of feature importance, particularly suited for high-dimensional datasets, where traditional feature importance measures (e.g., tree-based feature importances) might be biased or unreliable.

We used a 5-fold stratified cross-validation to ensure that each fold had an approximately equal proportion of CRC and healthy samples which is crucial to prevent class imbalance from affecting the model evaluation and the permutation importance estimates. For each feature in the validation set, we performed random shuffling of its values to disrupt any relationship with the target variable. The decrease in model performance was computed by comparing the model's performance on the original validation data versus the permuted data. The importance score for each feature was calculated as the mean decrease in performance over multiple shuffling iterations. We set the number of permutation-repeats to 5 (n_repeats=5) to obtain a reliable estimate of feature importance while managing computational cost. The importance scores from each fold were accumulated and averaged across all folds to obtain a stable estimate of each feature's importance.

After completing all folds, the accumulated importance scores for each feature were divided by the number of folds to compute the mean importance. The features were then ranked based on their mean importance scores in descending order, identifying the top contributing features to the model's predictive performance.

### 1.5. Statistical Analysis

All statistical analyses were conducted using R software (v.4.3.1). For each TCR feature, the expression levels between the HC and CRC groups were compared using the Wilcoxon rank-sum test, a non-parametric method suitable for comparing two independent groups without assuming normal distribution. Statistical significance was set at three levels: *p<0.05, **p<0.01 and *p<0.001.

Visualization was performed using the ggplot2 package. Boxplots were created for each feature to illustrate the distribution of expression levels in both groups. Significance levels were annotated on the plots using the ggsignif package.

### 2. Results

### 2.1. Comparison of TCR Repertoires Between CRC Patients and Healthy Controls

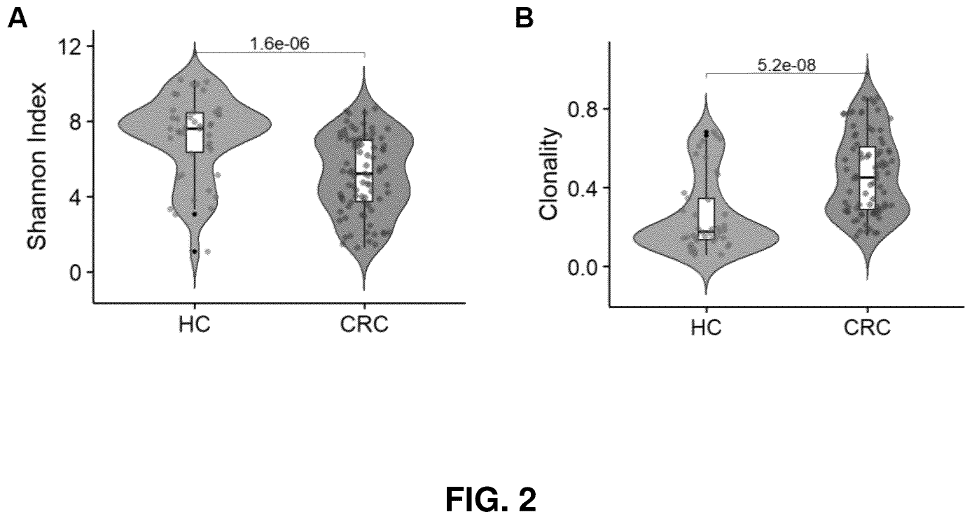

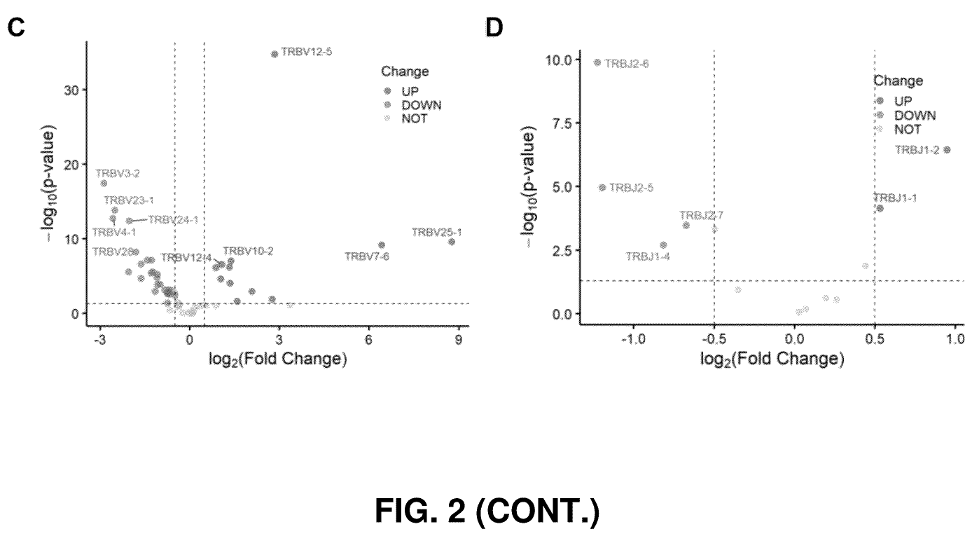

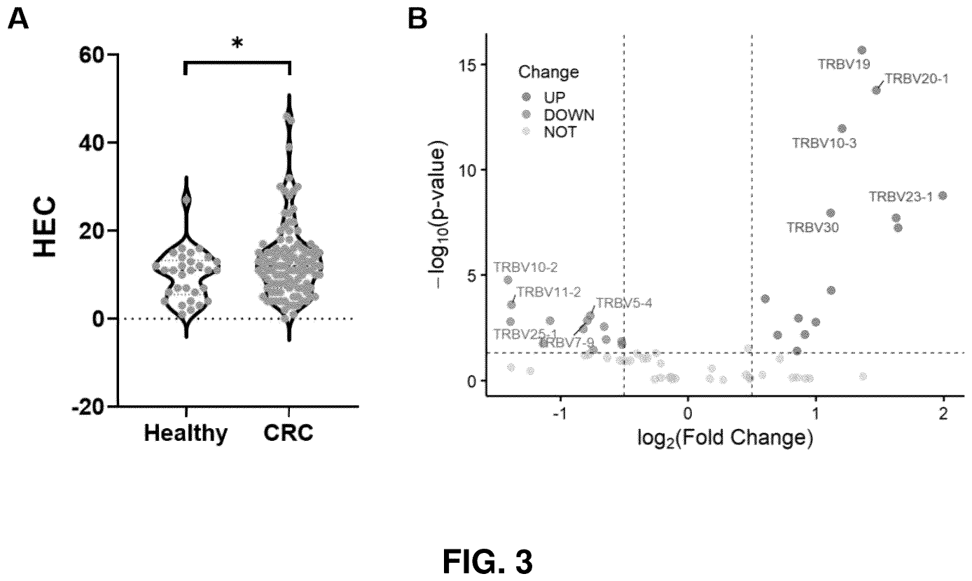

We analyzed the TCRβ repertoires in the peripheral blood of 42 healthy individuals and 83 CRC patients in the training and test sets [14, 16]. FIG. 2 provides a comprehensive analysis of TCR repertoire diversity, clonality, and differential Vβ and Jβ gene usage in CRC patients and healthy individuals. In FIG. 2, subplot A illustrates TCR Repertoire Shannon Diversity, each data point representing an individual sample; subplot B illustrates TCR repertoire Clonality, each data point representing an individual sample; subplot C depicts a Jβ gene usage volcano plot; and subplot D depicts a Vβ gene usage volcano plot (Wilcoxon test, p value <0.05 considered significant). FIG. 3 provides TCR repertoire analysis between CRC patients and healthy individuals. In FIG. 3, subplot A provides a comparison of HEC number distribution between healthy controls and CRC patients, *p<0.05, Mann-Whitney U test; and subplot B depicts a Vβ gene usage volcano plot.

Each sample exhibited distinct features in the TCR diversity. Healthy donors had relatively high TCR diversity and low TCR clonality (subplots A and B of FIG. 2). Furthermore, significantly higher usages of TRBV3-2, TRBV4-1, TRBV4-2, TRBV4-3, TRBV5-3, TRBV6-4, TRBV7-5, TRBV7-6, TRBV11-3, TRBV12-3, TRBV12-5, TRBV21-1, TRBV23-1, and TRBV24-1 were observed in healthy donors compared to CRC patients among the functional human Vβ genes. Conversely, significantly higher usages of TRBV2, TRBV3-1, TRBV5-1, TRBV5-5, TRBV5-6, TRBV5-8, TRBV6-3, TRBV7-2, TRBV7-3, TRBV7-7, TRBV7-8, TRBV7-9, TRBV9, TRBV10-1, TRBV10-2, TRBV12-4, TRBV14, TRBV15, TRBV16, TRBV20-1, TRBV25-1, TRBV28, TRBV29-1, and TRBV30 were observed in CRC patients compared to healthy donors (subplot C of FIG. 2, p<0.05, Mann-Whitney U test). Similarly, among the functional Jβ genes, significantly higher usages of TRBJ1-1, TRBJ1-4, TRBJ2-5, and TRBJ2-7 were observed in CRC patients compared to healthy donors, while significantly higher usages of TRBJ1-2 and TRBJ2-6 were observed in healthy donors (subplot D of FIG. 2, p<0.05, Mann-Whitney U test). We also conducted a comparative analysis on a data set of TCR repertoire of 107 CRC patients and 30 healthy individuals [15]. Another measure of immune diversity is the expression level of each unique clone. TCR clones with a frequency above 0.5% of total reads in a sample were defined as high-expansion clones [15].

Similar to findings reported in U.S. Provisional Patent Application No. 63/625,326, the comparison between the CRC and healthy groups revealed a significantly higher high-expansion clones' ratio in the CRC group (subplot A of FIG. 3). Additionally, we performed an analysis of differential functional human Vβ genes (subplot B of FIG. 3), which showed considerable overlap with previous findings. Specifically, the usages of TRBV5-5, TRBV7-7, TRBV7-8, TRBV7-9, TRBV10-1, TRBV10-2, TRBV29-1 were significantly higher, whereas lower levels of TRBV4-1, TRBV4-2, TRBV6-4, TRBV23-1, TRBV24-1, in CRC than in healthy individuals. These results demonstrate the consistency of the TCR repertoire across different CRC datasets and further substantiate the potential of the TCR repertoire as a biomarker for diagnosing CRC.

### 2.2. Performance of TCR Repertoire-Based ML Models for CRC Diagnosis

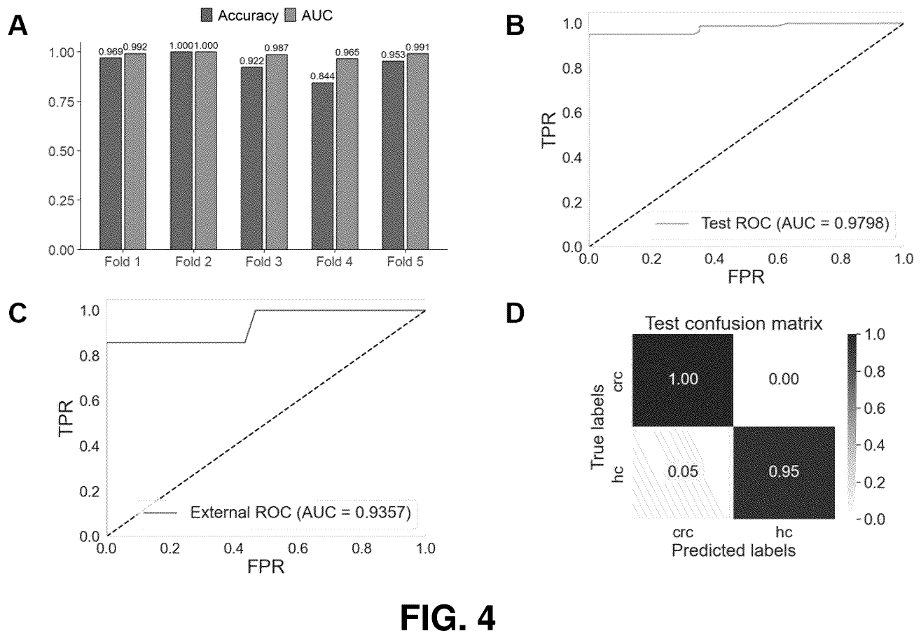

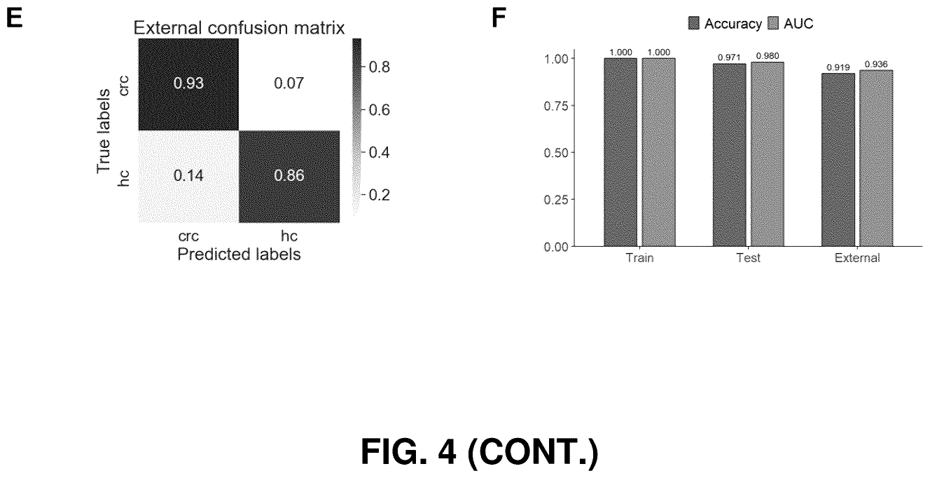

FIG. 4 illustrates performance of the RF models. In FIG. 4, subplot A plots the accuracy and AUC across five folds of cross-validation; subplot B plots the ROC curve for the test set with an AUC of 0.9798, in which the ROC curve plots the TPR against the FPR, demonstrating the model's diagnostic ability to distinguish between CRC and healthy individuals; subplot C plots the ROC curve for the external validation set with an AUC of 0.9357; subplot D depicts a confusion matrix for the test set, detailing the number of correct predictions (diagonal entries) versus incorrect predictions (off-diagonal entries); subplot E depicts the confusion matrix for the external validation set; and subplot F provides a comparative assessment of accuracy and AUC across training, test and external datasets.

Given that TCR diversity is primarily determined by the CDR3, which arises from the somatic recombination of V, D, and J gene segments, we constructed ML models using TRBV-TRBJ gene combination fraction tables as the primary features. To evaluate the performance of the diagnostic model, we performed a five-fold cross-validation. The AUC values for each fold were consistently high, ranging from 0.965 to 1.000, resulting in an average AUC of 0.9868 (subplot A of FIG. 4). This consistency indicates excellent discriminatory capability of the model.

The ROC curve in the test set achieved an AUC of 0.9798, further demonstrating the model's high predictive accuracy (subplot B of FIG. 4). The ROC curve for the external validation dataset yielded an AUC of 0.9357, confirming the robustness and generalizability of our TCR-based approach (subplot C of FIG. 4). To further assess the model's performance, we constructed confusion matrices for both the test and external validation datasets. In the test set, the model correctly classified 95% of CRC samples and 100% of healthy controls (subplot D of FIG. 4). Similarly, in the external validation set, the model accurately identified 93% of CRC cases and 86% of healthy individuals (subplot E of FIG. 4).

Finally, we conducted a comparative analysis of accuracy and AUC values across the training, test, and external validation sets (subplot F of FIG. 4). The consistently high-performance metrics across all datasets underscore the reliability of TCR repertoire markers for CRC diagnosis. In contrast to other artificial intelligence-assisted techniques for identifying CRC through blood tests [22], which consists of a binary SVM classifier at the first level and a one-class SVM classifier at the second level (achieving 60% sensitivity and 79% specificity), and the use of Red Cell Distribution Width [23], which demonstrated a sensitivity of 84% and a specificity of 88% for right-sided colon cancer, our model demonstrates superior performance.

In summary, these results demonstrate that TCR repertoire profiling, when analyzed through ML models, can serve as a highly accurate and robust diagnostic marker for CRC, presenting significant potential for clinical applications in early detection.

### 2.3. Identification of Key TCR Repertoire Biomarkers

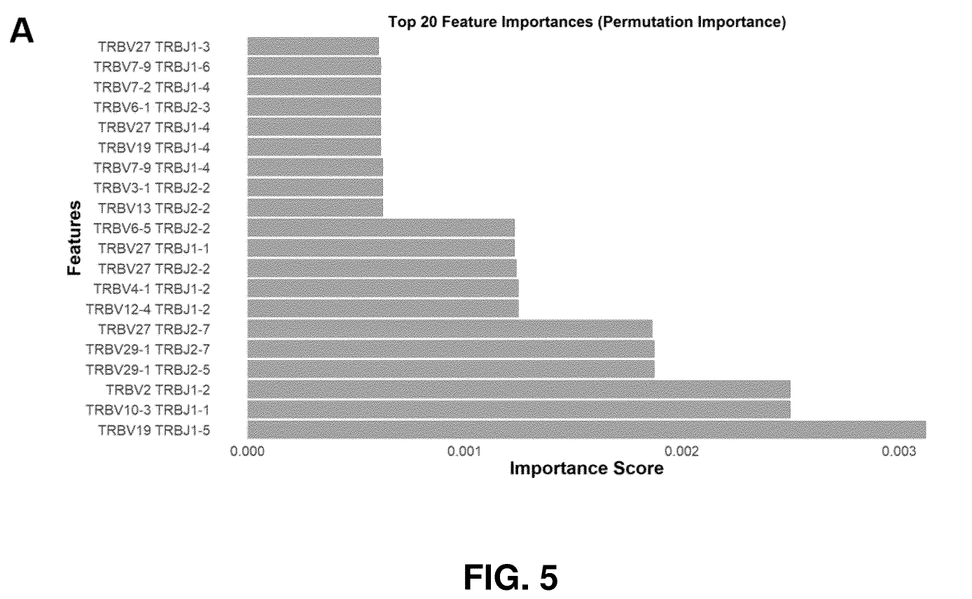

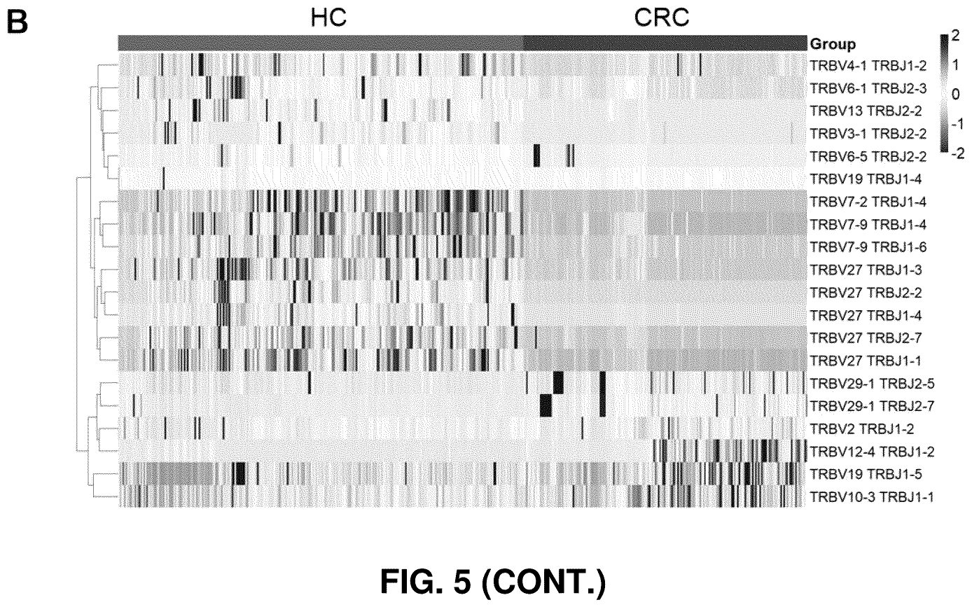

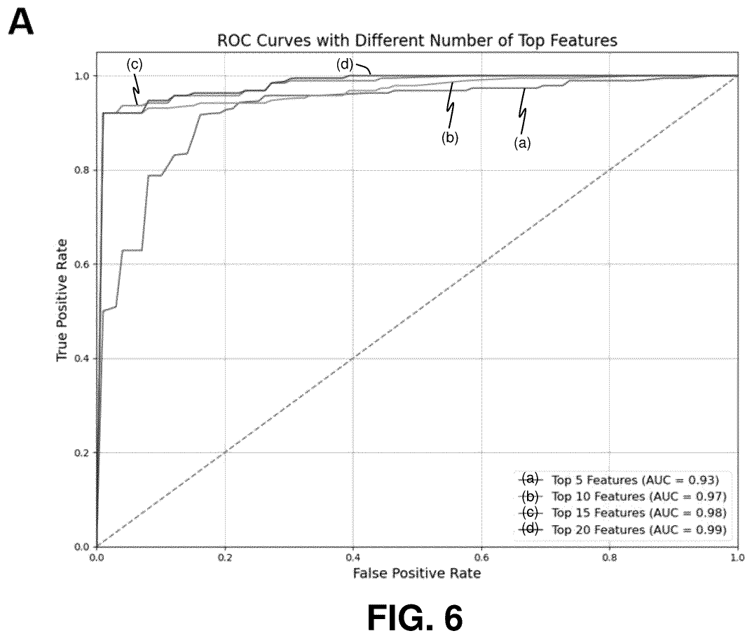

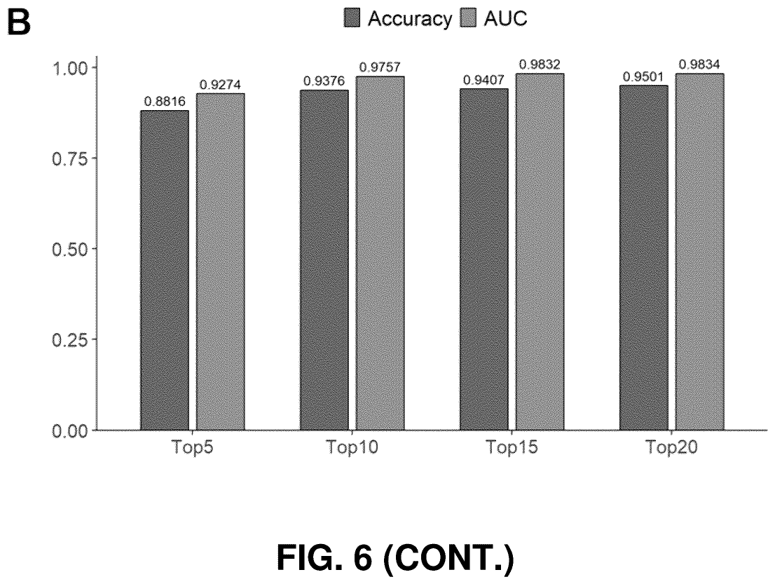

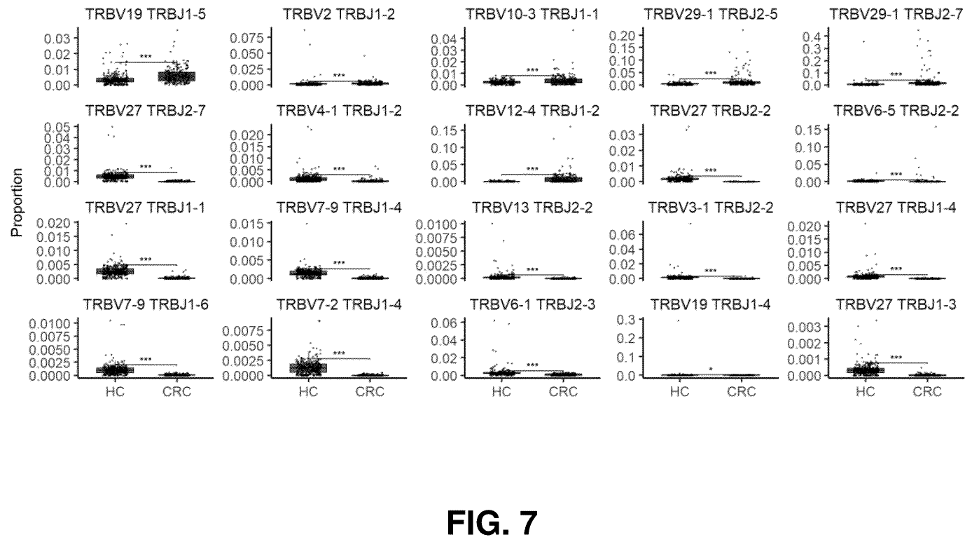

The identification of these biomarkers is illustrated with the aid of FIGS. 5-7. FIG. 5 illustrates identification of key features distinguishing CRC patients from healthy controls. In FIG. 5, subplot A lists the top 20 feature importances determined by permutation importance for the CRC diagnostic model, in which important scores quantify the impact of each feature on the model's predictive accuracy, with higher values indicating greater importance; and subplot B depicts a heatmap illustrating the expression levels of the top 20 features between CRC and healthy individuals. FIG. 6 provides performances of RF models employing different numbers of features in the test cohort. In FIG. 6, subplot A plots ROC curves for the test set with different numbers of features; and subplot B provides a comparative analysis of test set accuracy and AUC across ML models trained with different numbers of features. FIG. 7 provides a statistical analysis of the top 20 features. The top 20 features were analyzed using the Wilcoxon test (*p<0.05, **p<0.01, *p<0.001).

In our analysis to identify significant TCR repertoire biomarkers for CRC diagnosis, we employed a ML model enhanced by permutation importance to quantify the impact of individual TCR features on disease classification. Subplot A of FIG. 5 shows the top 20 TCR features ranked by their permutation importance scores. This list is dominated by specific combinations of TCR variable TRBV and TRBJ gene segments, with the highest importance score observed for TRBV19 TRBJ1-5, indicating its critical role in differentiating CRC from healthy controls.

We retrained the model using the top 5, top 10, top 15, and top 20 features and subsequently plotted the ROC curves (subplot A of FIG. 6). We also compared the accuracy and AUC on the test set across these four models (subplot B of FIG. 6). We found that the model trained with the top 20 features achieved the highest accuracy and AUC on the test set, recording values of 0.9501 and 0.9834, respectively. This result underscores the importance of the 20 features identified for the model.

To further investigate the expression patterns of these key TCR features across individual samples, we constructed a heatmap (subplot B of FIG. 5) that displays the expression levels of the top 20 TCR features in both HCs and CRC patients. The heatmap reveals distinct clustering of samples, with specific TCR features demonstrating differential expression between the two groups. Notably, features such as TRBV19 TRBJ1-5 and TRBV10-3 TRBJ1-1 exhibited higher expression levels predominantly in CRC samples as compared to HCs, highlighting their potential as biomarkers.

The differential expression of these TCR features was statistically significant (FIG. 7), with all features showing adjusted p-values <0.05, confirming their relevance in CRC pathology. This analysis not only underscores the utility of TCR repertoire analysis for identifying potential diagnostic markers but also suggests that certain combinations of TCR gene segments may be intimately involved in the immune response to CRC.

These findings provide a valuable foundation for further investigations into the TCR repertoire in CRC and may contribute to the development of novel diagnostic and therapeutic strategies targeting specific TCR interactions.

### 2.4. Performance Comparison of ML Methods in CRC Diagnosis

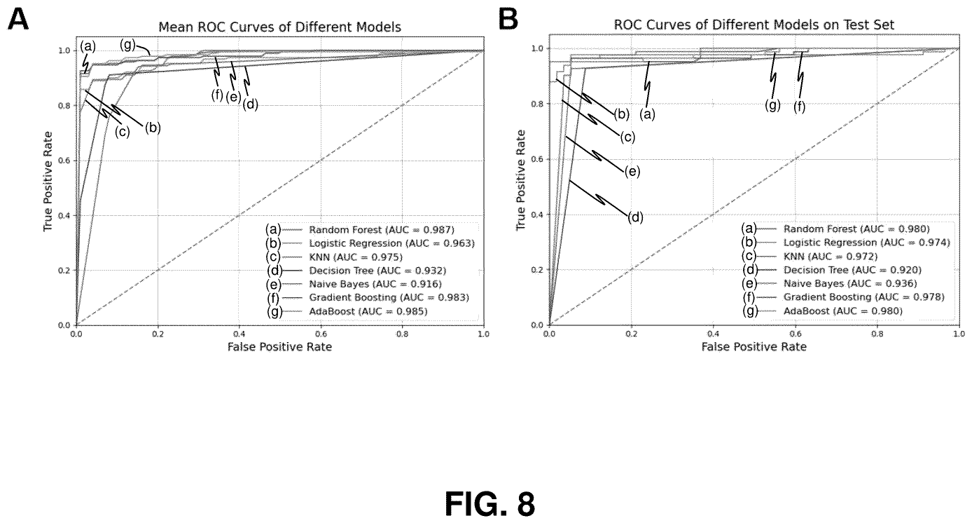

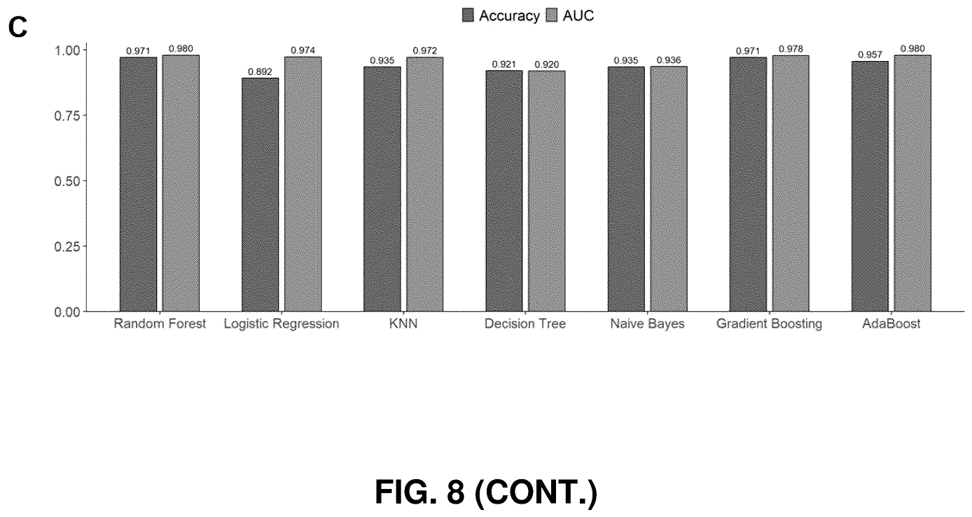

To evaluate the effectiveness of various ML algorithms in diagnosing CRC based on TCR repertoire data, we assessed seven different models: RF, LR, KNN, DT, Naive Bayes, Gradient Boosting, and AdaBoost. FIG. 8 provides performances of seven ML methods in TCR repertoire-based disease classification. In FIG. 8, subplot A plots the mean ROC curves for the validation sets across the seven ML methods; subplot B plots the ROC curves for the test set across the seven ML methods; and subplot C provides a comparative analysis of test set accuracy and AUC across the seven ML methods.

As illustrated in subplot A of FIG. 8, the mean ROC curves obtained from cross-validation demonstrate that the RF model achieved the highest overall performance, with an AUC value of 0.987.

We further validated the performance of these models on an independent test set (subplot B of FIG. 8). The results indicate that the RF model maintained superior performance, achieving an AUC of 0.980. Subplot C of FIG. 8 presents a detailed comparison of the accuracy and AUC values for each model. Notably, the RF consistently outperformed the other methods, with an accuracy of 0.971 and an AUC of 0.980.

In summary, our findings demonstrate that ML methods, particularly the RF algorithm, can accurately diagnose CRC based on TCR repertoire data. These results highlight the potential of utilizing TCR profiling in conjugation with ML algorithms as a non-invasive diagnostic tool for CRC. The accuracy and AUC of the validation and test sets across seven ML methods are illustrated in Table 2 and Table 3, all indicating the superior performance of RF.

TABLE 2
Accuracy of the validation and test sets across seven ML methods.

| ML model | Fold 1 | Fold 2 | Fold 3 | Fold 4 | Fold 5 | Average | Test |
|---|---|---|---|---|---|---|---|
| RF | 0.9692 | 1.0000 | 0.9219 | 0.8438 | 0.9531 | 0.9376 | 0.9712 |
| LR | 0.8923 | 0.9538 | 0.8750 | 0.8281 | 0.8594 | 0.8817 | 0.8921 |
| KNN | 0.8923 | 0.9538 | 0.9375 | 0.8594 | 0.8750 | 0.9036 | 0.9353 |
| DT | 0.9385 | 0.9846 | 0.9375 | 0.8281 | 0.9219 | 0.9221 | 0.9209 |
| Naive | 0.9231 | 0.9385 | 0.9375 | 0.8594 | 0.9219 | 0.9161 | 0.9353 |
| Bayes | | | | | | | |
| Gradient | 0.9846 | 0.9846 | 0.9688 | 0.8594 | 0.9375 | 0.9470 | 0.9712 |
| Boosting | | | | | | | |
| AdaBoost | 0.9846 | 0.9846 | 0.9375 | 0.8750 | 0.9219 | 0.9407 | 0.9568 |

TABLE 3
AUC of the validation and test sets across seven ML methods.

| ML model | Fold 1 | Fold 2 | Fold 3 | Fold 4 | Fold 5 | Average | Test |
|---|---|---|---|---|---|---|---|
| RF | 0.9922 | 1.0000 | 0.9868 | 0.9646 | 0.9905 | 0.9868 | 0.9801 |
| LR | 0.9610 | 0.9942 | 0.9848 | 0.8917 | 0.9830 | 0.9629 | 0.9737 |
| KNN | 0.9825 | 0.9873 | 0.9939 | 0.9413 | 0.9710 | 0.9752 | 0.9720 |
| DT | 0.9717 | 0.9859 | 0.9352 | 0.8431 | 0.9224 | 0.9317 | 0.9196 |
| Naive | 0.9191 | 0.9357 | 0.9231 | 0.8917 | 0.9119 | 0.9163 | 0.9365 |
| Bayes | | | | | | | |
| Gradient | 0.9961 | 1.0000 | 0.9990 | 0.9271 | 0.9930 | 0.9830 | 0.9784 |
| Boosting | | | | | | | |
| AdaBoost | 0.9912 | 0.9990 | 0.9909 | 0.9555 | 0.9900 | 0.9853 | 0.9797 |

### 3. Embodiments of Present Disclosure

Embodiments of the present disclosure are developed as follows based on the details, examples, applications, etc. regarding the diagnostic model as disclosed above possibly with generalization and extension.

An aspect of the present disclosure is to provide a computer-implemented method for diagnosing CRC for a person.

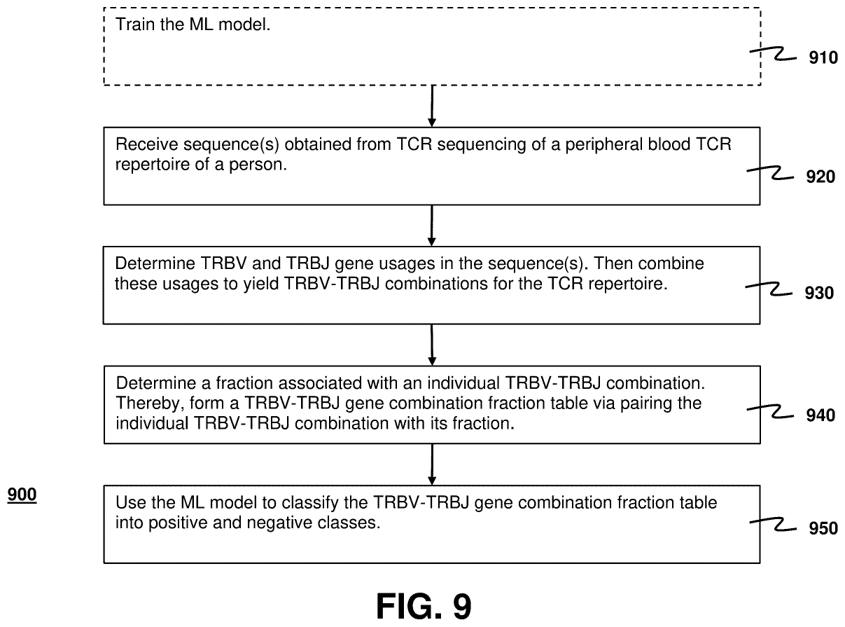

The disclosed method is illustrated with the aid of FIG. 9, which depicts a workflow 900 showing exemplary steps of the disclosed method. The disclosed method is related to operating a ML model. Exemplarily, the disclosed method comprises steps 920, 930, 940 and 950.

In the step 920, one or more sequences obtained from TCR sequencing of a peripheral blood TCR repertoire of the person are received.

The received one or more sequences are analyzed in the step 930 to determine TRBV and TRBJ gene usages in the one or more sequences. The TRBV and TRBJ gene usages as determined are combined to yield a plurality of TRBV-TRBJ combinations for the TCR repertoire. The plurality of TRBV-TRBJ combinations is a plurality of V-J gene combination features representing pairing of V and J gene segments of TCR β-chain of the TCR repertoire.

In the step 940, a fraction associated with an individual TRBV-TRBJ combination identified in the plurality of TRBV-TRBJ combinations is determined, where the fraction indicates a relative abundance of the individual TRBV-TRBJ combination in the one or more sequences. Also in the step 940, a TRBV-TRBJ gene combination fraction table for the plurality of TRBV-TRBJ combinations is formed via pairing the individual TRBV-TRBJ combination with the fraction associated therewith. As a result, the TRBV-TRBJ gene combination fraction table has a plurality of entries. An individual entry lists a certain TRBV-TRBJ combination and a corresponding fraction associated with this certain TRBV-TRBJ combination. This certain TRBV-TRBJ combination is identified in the plurality of TRBV-TRBJ combinations, and the TRBV-TRBJ gene combination fraction table lists respective entries for all TRBV-TRBJ combinations found in the plurality of TRBV-TRBJ combinations.

In the step 950, a ML model is employed to classify the TRBV-TRBJ gene combination fraction table into a positive class and a negative class after the ML model is trained. The positive class indicates that the person is diagnosed with CRC while the negative class indicates that the person is not diagnosed with CRC.

An individual sequence among the received one or more sequences may be any sequence obtained from TCR sequencing of the peripheral blood TCR repertoire. In practice, the individual sequence as received is usually a CDR3 sequence.

Preferably, the ML model is realized as a RF model.

Apart from the RF model, other networks may be used for the ML model. The ML model may be selected from a LR model, a KNN model, a Naive Bayes model, a DT model, a gradient boosting model, an AdaBoost model, etc.

Although seven ML models are considered in Sections 1 and 2 for generating experimental results, the present disclosure is not limited only to selecting one of these seven ML models as the ML model of the disclosed method. As an example, a transformer may be selected to be the ML model for certain advantages that will soon be realized.

As mentioned above, key TCR repertoire biomarkers for CRC are identified in the present disclosure. One may focus on these key biomarkers in analyzing the TCR repertoire of the person for CRC diagnosis to achieve certain advantages, e.g., a shorter execution time to diagnose CRC, and a possibility of using a lightweight portable device for CRC pre-screening in poorly developed areas. In certain embodiments, the ML model is configured with an attention mechanism to focus on a plurality of selected TRBV-TRBJ gene combinations in processing the TRBV-TRBJ gene combination fraction table. The plurality of selected TRBV-TRBJ gene combinations is formed with the key TCR repertoire biomarkers for CRC as identified above. It follows that the plurality of selected TRBV-TRBJ gene combinations consists of combinations of: TRBV19 and TRBJ1-5; TRBV10-3 and TRBJ 1-1; TRBV2 and TRBJ1-2; TRBV29-1 and TRBJ 2-5; TRBV29-1 and TRBJ2-7; TRBV27 and TRBJ2-7; TRBV12-4 and TRBJ1-2; TRBV4-1 and TRBJ1-2; TRBV27 and TRBJ2-2; TRBV27 and TRBJ1-1; TRBV6-5 and TRBJ2-2; TRBV13 and TRBJ2-2; TRBV3-1 and TRBJ2-2; TRBV7-9 and TRBJ1-4; TRBV19 and TRBJ1-4; TRBV27 and TRBJ1-4; TRBV6-1 and TRBJ2-3; TRBV7-2 and TRBJ1-4; TRBV7-9 and TRBJ1-6; and TRBV27 and TRBJ1-3.

Many ML networks, such as a transformer, may be used as the above-mentioned ML model configured with the attention mechanism. In certain embodiments, the ML model is selected to be a transformer.

Note that the step 950 is executed only after the ML model is trained. The ML model may be trained prior to execution of the workflow 900 by, for instance, loading pre-stored model parameters into the ML model where the pre-stored model parameters have been obtained through training the ML model. Alternatively, the ML model may be trained during the execution of the workflow 900. In certain embodiments, the workflow 900 further comprises a step of training the ML model, namely, step 910, where the step 910 is executed prior to the execution of the step 950.

A computing system for diagnosing CRC for a person is realizable by including one or more computers, where the one or more computers are configured to execute a process of diagnosing CRC for the person according to any of the embodiments of the disclosed method. An individual computer may be a general-purpose computer, a special-purpose computer such as the one implemented with artificial intelligence processor(s) or graphics processing unit(s), a desktop computer, a physical computing server, a distributed computing server, or a mobile computing device such as a smartphone and a tablet computer.

The present disclosure may be embodied in other specific forms without departing 1 from the spirit or essential characteristics thereof. The present embodiment is therefore to be considered in all respects as illustrative and not restrictive. The scope of the invention is indicated by the appended claims rather than by the foregoing description, and all changes that come within the meaning and range of equivalency of the claims are therefore intended to be embraced therein.

### REFERENCES

There follows a list of references that are occasionally cited in the specification. Each of the disclosures of these references is incorporated by reference herein in its entirety.
1. Rawla, P., T. Sunkara, and A. Barsouk, Epidemiology of colorectal cancer: incidence, mortality, survival, and risk factors. Gastroenterology Review/Przeglgd Gastroenterologiczny, 2019. 14(2): p. 89-103.
2. Bell, C. C. and O. Gilan, Principles and mechanisms of non-genetic resistance in cancer. British journal of cancer, 2020. 122(4): p. 465-472.
3. Mármol, I., et al., Colorectal carcinoma: a general overview and future perspectives in colorectal cancer. International journal of molecular sciences, 2017. 18(1): p. 197.
4. Lin, J. S., et al., Screening for colorectal cancer: updated evidence report and systematic review for the US Preventive Services Task Force. Jama, 2016. 315(23): p. 2576-2594.
5. IJsselsteijn, M. E., et al., Colorectal cancer: a paradigmatic model for cancer immunology and immunotherapy. Molecular aspects of medicine, 2019. 69: p. 123-129.
6. Jia, Q., et al., Diversity index of mucosal resident T lymphocyte repertoire predicts clinical prognosis in gastric cancer. Oncoimmunology, 2015. 4(4): p. e1001230.
7. Han, Y., et al., Identification of characteristic TRB V usage in HBV-associated HCC by using differential expression profiling analysis. Oncoimmunology, 2015. 4(8): p. e1021537.
8. Postow, M. A., et al., Peripheral T cell receptor diversity is associated with clinical outcomes following ipilimumab treatment in metastatic melanoma. Journal for immunotherapy of cancer, 2015. 3(1): p. 1-5.
9. Rahman, S. F., et al., Machine learning leveraging genomes from metagenomes identifies influential antibiotic resistance genes in the infant gut microbiome. MSystems, 2018. 3(1): p. 10.1128/msystems. 00123-17.
10. Cammarota, G., et al., Gut microbiome, big data and machine learning to promote precision medicine for cancer. Nature reviews gastroenterology & hepatology, 2020. 17(10): p. 635-648.
11. Su, Q., et al., Faecal microbiome-based machine learning for multi-class disease diagnosis. Nature Communications, 2022. 13(1): p. 6818.
12. Manandhar, I., et al., Gut microbiome-based supervised machine learning for clinical diagnosis of inflammatory bowel diseases. American Journal of Physiology-Gastrointestinal and Liver Physiology, 2021. 320(3): p. G328-G337.
13. Katayama, Y., et al., Machine learning approaches to TCR repertoire analysis. Frontiers in Immunology, 2022. 13: p. 858057.
14. Chen, Y.-T., et al., Longitudinal high-throughput sequencing of the T-cell receptor repertoire reveals dynamic change and prognostic significance of peripheral blood TCR diversity in metastatic colorectal cancer during chemotherapy. Frontiers in Immunology, 2022. 12: p. 743448.
15. Cao, Y., et al., Colorectal cancer-associated T cell receptor repertoire abnormalities are linked to gut microbiome shifts and somatic cell mutations. Gut Microbes, 2023. 15(2): p. 2263934.
16. Malik, A., et al., The role of CD8+ T-cell clones in immune thrombocytopenia. Blood, The Journal of the American Society of Hematology, 2023. 141(20): p. 2417-2429.
17. Rosati, E., et al., A novel unconventional T cell population enriched in Crohn's disease. Gut, 2022. 71(11): p. 2194-2204.
18. Von Niederhäusern, V., M. Ghraichy, and J. Truck, Applicability of T cell receptor repertoire sequencing analysis to unbalanced clinical samples-comparing the T cell receptor repertoire of GATA2 deficient patients and healthy controls. Swiss Medical Weekly, 2023. 153(2): p. 40046-40046.
19. Bolotin, D. A., et al., MiXCR: software for comprehensive adaptive immunity profiling. Nature methods, 2015. 12(5): p. 380-381.
20. Uddin, S., et al., Comparing different supervised machine learning algorithms for disease prediction. BMC medical informatics and decision making, 2019. 19(1): p. 1-16.
21. Bansal, M., A. Goyal, and A. Choudhary, A comparative analysis of K-nearest neighbor, genetic, support vector machine, decision tree, and long short term memory algorithms in machine learning. Decision Analytics Journal, 2022. 3: p. 100071.
22. Soares, F., K. Becker, and M. J. Anzanello, A hierarchical classifier based on human blood plasma fluorescence for non-invasive colorectal cancer screening. Artificial Intelligence in Medicine, 2017. 82: p. 1-10.
23. Nogueira-Rodríguez, A., et al., Deep neural networks approaches for detecting and classifying colorectal polyps. Neurocomputing, 2021. 423: p. 721-734.
24. Qi, Y., Random forest for bioinformatics. Ensemble machine learning: Methods and applications, 2012: p. 307-323.
25. Charbuty, B. and A. Abdulazeez, Classification based on decision tree algorithm for machine learning. Journal of Applied Science and Technology Trends, 2021. 2(01): p. 20-28.
26. Boyko, N. and K. Boksho, Application of the Naive Bayesian Classifier in Work on Sentimental Analysis of Medical Data. IDDM, 2020. 2020: p. 3rd.
27. Ning, Y., et al. Fall detection algorithm based on gradient boosting decision tree. in 2019 IEEE International Conference on Signal Processing, Communications and Computing (ICSPCC). 2019. IEEE.
28. Ding, Y., et al., An efficient AdaBoost algorithm with the multiple thresholds classification. Applied sciences, 2022. 12(12): p. 5872.
29. Dou, B., et al., Machine learning methods for small data challenges in molecular science. Chemical Reviews, 2023. 123(13): p. 8736-8780.

What is claimed is:

1. A computer-implemented method for diagnosing colorectal cancer (CRC) for a person, the method comprising:
receiving one or more sequences obtained from T cell receptor (TCR) sequencing of a peripheral blood TCR repertoire of the person;
determining TRBV and TRBJ gene usages in the one or more sequences;
combining the TRBV and TRBJ gene usages to yield a plurality of TRBV-TRBJ combinations for the TCR repertoire, the plurality of TRBV-TRBJ combinations being a plurality of V-J gene combination features representing pairing of V and J gene segments of TCR β-chain of the TCR repertoire;
determining a fraction associated with an individual TRBV-TRBJ combination, wherein the fraction indicates a relative abundance of the individual TRBV-TRBJ combination in the one or more sequences;
forming a TRBV-TRBJ gene combination fraction table for the plurality of TRBV-TRBJ combinations via pairing the individual TRBV-TRBJ combination with the fraction associated therewith; and
using a machine learning (ML) model to classify the TRBV-TRBJ gene combination fraction table into a positive class and a negative class after the ML model is trained, the positive class indicating that the person is diagnosed with CRC, the negative class indicating that the person is not diagnosed with CRC.

2. The method of claim 1, wherein the ML model is configured with an attention mechanism to focus on a plurality of selected TRBV-TRBJ gene combinations in processing the TRBV-TRBJ gene combination fraction table, wherein the plurality of selected TRBV-TRBJ gene combinations consists of combinations of: TRBV19 and TRBJ1-5; TRBV10-3 and TRBJ 1-1; TRBV2 and TRBJ1-2; TRBV29-1 and TRBJ 2-5; TRBV29-1 and TRBJ2-7; TRBV27 and TRBJ2-7; TRBV12-4 and TRBJ1-2; TRBV4-1 and TRBJ1-2; TRBV27 and TRBJ2-2; TRBV27 and TRBJ1-1; TRBV6-5 and TRBJ2-2; TRBV13 and TRBJ2-2; TRBV3-1 and TRBJ2-2; TRBV7-9 and TRBJ1-4; TRBV19 and TRBJ1-4; TRBV27 and TRBJ1-4; TRBV6-1 and TRBJ2-3; TRBV7-2 and TRBJ1-4; TRBV7-9 and TRBJ1-6; and TRBV27 and TRBJ1-3.

3. The method of claim 1, wherein each of the received one or more sequences is a complementarity determining region 3 (CDR3) sequence.

4. The method of claim 1, wherein the ML model is a random forest (RF) model.

5. The method of claim 1, wherein the ML model is selected from a logistic regression (LR) model, a K-nearest neighbors (KNN) model, a Naive Bayes model, a decision tree (DT) model, a gradient boosting model and an AdaBoost model.

6. The method of claim 1, wherein the ML model is a transformer.

7. The method of claim 1 further comprising training the ML model.

8. A computing system for diagnosing colorectal cancer (CRC) for a person, the computing system comprising one or more computers configured to execute a process of diagnosing CRC for the person according to the method of claim 1.

9. A computing system for diagnosing colorectal cancer (CRC) for a person, the computing system comprising one or more computers configured to execute a process of diagnosing CRC for the person according to the method of claim 2.

10. A computing system for diagnosing colorectal cancer (CRC) for a person, the computing system comprising one or more computers configured to execute a process of diagnosing CRC for the person according to the method of claim 3.

11. A computing system for diagnosing colorectal cancer (CRC) for a person, the computing system comprising one or more computers configured to execute a process of diagnosing CRC for the person according to the method of claim 4.

12. A computing system for diagnosing colorectal cancer (CRC) for a person, the computing system comprising one or more computers configured to execute a process of diagnosing CRC for the person according to the method of claim 5.

13. A computing system for diagnosing colorectal cancer (CRC) for a person, the computing system comprising one or more computers configured to execute a process of diagnosing CRC for the person according to the method of claim 6.

14. A computing system for diagnosing colorectal cancer (CRC) for a person, the computing system comprising one or more computers configured to execute a process of diagnosing CRC for the person according to the method of claim 7.
